(19) United States
(12) Patent Application Publication
(10) Pub. No.: US 2009/0203997 A1
(43) Pub. Date: Aug. 13, 2009

(54) ULTRASOUND DISPLACEMENT IMAGING WITH SPATIAL COMPOUNDING

(76) Inventor: Kutay Ustuner, Mountain View, CA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/027,957

(22) Filed: Feb. 7, 2008

(51) Int. Cl. A61B 8/00

(52) U.S. Cl. 600/443; 382/128

(57) ABSTRACT

Artifacts in ultrasound displacement images are reduced by combining multiple component displacement images. For each component displacement image first a pre-displacement ultrasound image is generated from a particular imaging angle. Then a displacement force is applied on the object at a desired displacement angle via an ultrasound or other mechanical force. Then a post-displacement ultrasound image is generated from the same imaging angle. A component displacement image is generated by correlating the pre-displacement and post-displacement ultrasound images. The above steps are repeated for at least one other (imaging angle, displacement angle) pair, and the resulting component displacement images are combined to reduce displacement image artifacts.

### BACKGROUND

The present embodiments relate to ultrasound imaging. In particular, images of tissue displacement are generated with ultrasound scanning.

The first step of ultrasound displacement imaging is to generate a pre-displacement ultrasound image from a particular imaging angle. Then a displacement force is applied on the object at a desired displacement angle via an ultrasound or other mechanical means. Then a post-displacement ultrasound image is generated from the same imaging angle. A displacement image is generated by correlating the pre-displacement and post-displacement ultrasound images. Techniques such as Elastography, ARFI (Acoustic Radiation Force Imaging), strain and strain rate are various forms of displacement imaging techniques.

One ultrasound displacement imaging mode is elasticity imaging. U.S. Pat. Nos. 5,107,837; 5,293,870; 5,178,147; and 6,508,768 describe methods to generate elasticity images using the relative tissue displacement between adjacent frames. The displacement force is applied by pressing on the skin surface. For example, the sonographer presses the transducer against the patient. A device, such as a plate or transducer, may apply the force. U.S. Pat. No. 6,558,324 describes methods to represent elasticity using color coding.

For displacement imaging, pressure is applied to stress internal tissue. The response of the internal tissue to the application or release of the stress is measured with ultrasound energy. For example, correlation of B-mode data representing the tissue under different stress loads is used to determine tissue displacement. The displacement data includes the strain, a strain rate, modulus, or other parameter corresponding to the tissue displacement. The displacement may indicate a lesion. Lesions may have stiffer tissue than the surrounding healthy tissue.

For cardiac imaging, the displacement rate may be determined using the heart motion as the source of stress. Stress may be applied acoustically. Acoustic radiation force imaging (ARFI) exploits the stiffness difference between a lesion and surrounding tissues. For example, see U.S. Pat. No. 6,371,912, the disclosure of which is incorporated herein by reference. The radiation force of a strong pushing pulse induces micron level displacement of the target area. Two-dimensional speckle tracking provides displacement over a millisecond period of tissue movement.

Spatial variations in the object's mechanical properties introduce hard to model variations in the spatial distribution of the displacement force. This reduces the signal-to-noise ratio (SNR) of the displacement image and the accuracy of the displacement-based estimates, such as strain. Similarly spatial variations in the speed of sound across refractive interfaces or aberrating regions, and highly attenuative or reflective tissue cause shadowing, defocusing or hard to model geometric distortions in the pre- and post-displacement ultrasound images. This affects the SNR and spatial accuracy of the ultrasound images. In ARFI, the displacement force spatial nonuniformity may also be caused by the radiation force source due to transmit focusing and the finite extent of the transmit depth of field.

If the deposited acoustical/mechanical energy varies within tissue, the amount of displacement varies as well, causing artifacts in the displacement images. One can't tell, for example, if a dark area in an ARFI image is due to a refraction shadow, attenuation shadow or stiffer tissue.

### BRIEF SUMMARY

By way of introduction, the preferred embodiments described below include methods, instructions, computer readable media, and systems for ultrasound-based displacement imaging with reduced artifacts. Artifacts may be reduced by combining different frames of displacement data. Each frame of displacement data is determined from two or more component frames of data (e.g., correlating B-mode data from scans of the same region under different pressure). The displacement frames have a different displacement force or imaging angle, but represent the same region. By combining displacement data associated with different angles, the effect of artifacts may be reduced.

In a first aspect, a method is provided for ultrasound-based displacement imaging with reduced artifacts. A first frame of displacement data is acquired with ultrasound for a first region. The first region corresponds to a first position of a transducer. The displacement data of the first frame is responsive to a first angle. A second frame of displacement data is acquired with ultrasound for the first region corresponding to the first position of the transducer. The displacement data of the second frame is responsive to a second angle different than the first angle. For each of a plurality of spatial locations of the first region, the displacement data of the first frame is combined with the displacement data of the second frame. An image of the first region is generated as a function of the combined displacement data.

In a second aspect, a computer readable storage medium has stored therein data representing instructions executable by a programmed processor for ultrasound-based displacement imaging with reduced artifacts. The storage medium includes instructions for forming tissue displacement frames of data in response to different displacement force angles, the tissue displacement frames of data representing a same region, and generating an image of the region as a function of the tissue displacement frames of data.

In a third aspect, a method is provided for ultrasound-based displacement imaging with reduced artifacts. A transducer is positioned adjacent a region to be imaged. First acoustic force is transmitted from the transducer at a first group of one or more angles relative to the transducer. A first displacement of tissue in the region responsive to the first acoustic force is determined. A second acoustic force from the transducer at a second group of one or more angles relative to the transducer is transmitted. The one or more angles of the second group are different than any of the one or more angles of the first group. A second displacement of the tissue in the region responsive to the second acoustic force is determined. For each spatial location in the region, the first and second displacements are combined. The first and second groups correspond to scan lines for scanning the entire region. An image of the region is generated as a function of the combined first and second displacements for each spatial location.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for ultrasound-based displacement imaging with reduced artifacts;

FIG. 2 is a representation of scan lines for steered compound displacement imaging; and

FIG. 3 is a block diagram of one embodiment of a system for ultrasound-based displacement imaging with reduced artifacts.

### DETAILED DESCRIPTION OF THE DRAWINGS AND PRESENTLY PREFERRED EMBODIMENTS

To reduce displacement force nonuniformity artifacts, multiple displacement images are combined. Artifacts in ultrasound displacement images are reduced by combining multiple component displacement images associated with different angles, foci, and/or frequencies. For each component displacement image, first a pre-displacement ultrasound image is generated from a particular imaging angle. Then a displacement force is applied on the object at a desired displacement angle via an ultrasound or other mechanical force. Then a post-displacement ultrasound image is generated from the same imaging angle. A component displacement image is generated by correlating the pre-displacement and post-displacement ultrasound images. The above steps are repeated for at least one other (imaging angle, displacement angle) pair, and the resulting component displacement images are combined to reduce displacement image artifacts.

Spatial variance may provide more accurate representation of displacement response of tissue. Typically, steered spatial compounding has the undesired result of losing clinical markers. However, displacement imaging may be more useful without the markers.

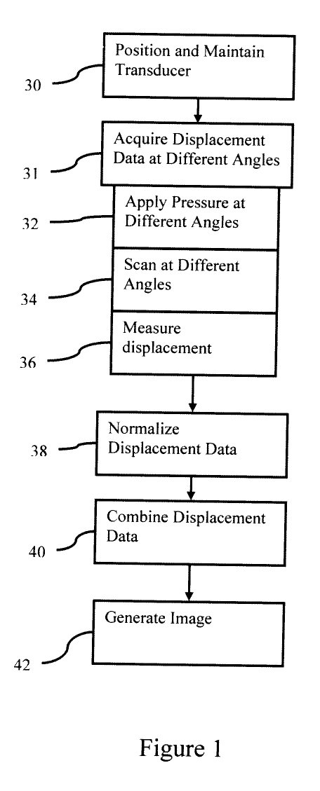

FIG. 1 shows a method for ultrasound-based displacement imaging with reduced artifacts. In some embodiments, the method is for elasticity ultrasound imaging. In other embodiments, the method is for acoustic force radiation imaging. Additional, different or fewer acts may be provided. For example, act 32 or act 34 is optional. The different angles may be applied to the application of displacement force and/or the scanning to determine displacement. As another example, the imaging act 42 or normalizing act 38 are not performed. The acts are performed in the order described or shown, but other orders may be provided.

In act 30, the transducer is positioned adjacent a region to be imaged. B-mode or other ultrasound imaging may be performed prior to displacement imaging. The user identifies a region of interest by moving the transducer and making any adjustments to the imaging parameters (e.g., changing an imaging depth, scan format, and/or scan boundaries). A processor may assist or identify the region of interest.

Once the region to be imaged for displacement is identified, the transducer is maintained at the same position. Due to patient or sonographer motion, some movement of the transducer relative to the region may occur while maintaining the transducer at the same position. Either the user or a mechanical structure maintains the transducer at the position to scan the region of interest. The transducer is maintained at the position for transmitting the displacement force, transmitting and receiving (i.e., scanning) with ultrasound to measure displacement, and/or determining the displacement.

In act 31, different frames of displacement data are acquired with ultrasound. The displacement data is for the region of interest associated with the transducer position. The region of interest may be the entire scan region or a portion of the scan region. Since the different frames may be associated with different pressure or scan angles, the different frames cover substantially all of the same region, but may not overlap at portions due to steering. Acts 32, 34, and 36 are performed to acquire a frame of displacement data.

In act 32, different amounts of displacement pressure are applied to the region of interest. The different amounts may include two or more pressure levels for creating displacement, such as no pressure and a peak pressure. The displacement pressure is applied to the region of interest. The pressure is applied from different directions or a same direction at different times. The location of the source of pressure is at the transducer, is the transducer, straddles the transducer, surrounds the transducer, is adjacent to the transducer, and/or is spaced from the transducer.

The displacement is applied acoustically, such as associated with acoustic force radiation, or mechanically, such as associated with elasticity imaging. Displacement data may be generated with manual palpation, external vibration sources, inherent tissues motion (e.g., motion due to cardiac pulsations, or breathing) or acoustic radiation force imaging (ARFI). ARFI produces displacement images or produces relaxation images. The acoustic force may be provided by therapeutic ultrasound transmissions. The acoustic force may be used as a transmission for the scanning of act 34 or is a separate transmission.

In act 34, the region of interest is scanned to measure displacement. The region is scanned while subject to different amounts of displacement pressure. For example, scanning occurs while applying different amounts of acoustic pressure, such as with and without the acoustic radiation pressure. As another example, scanning occurs while applying different amounts of pressure with the transducer against a patient while the transducer is maintained in the first position. Elastography frames of data are formed using a manual or non-acoustic external force source.

For acquiring a frame of displacement data, at least two scans are performed. Scanning includes transmitting and receiving along one or more scan lines. Radio frequency data is received. The data is responsive to ultrasound transmissions and echoes. The radio frequency data is beamformed or represents different spatial locations scanned with ultrasound. Data from two or more scans of the same region is acquired with the transducer in the maintained position. The scans are repeated with a same scan line format. More than two frames of data may be acquired. Each frame of data represents a same two or three-dimensional region, such as associated with a complete scan or the transducer being generally at a same location. For three-dimensional imaging, a plurality of two-dimensional scans may represent the volume.

In one embodiment, the displacement force source may act as the transmitter for imaging as well (e.g., a high power transmission to generate the radiation force, followed by low power transmission for imaging), but not as the receiver. The imaging angle (round-trip) for this case is the mid way between the transmit and receive axes.

In act 36, displacement is measured. The displacement data is an estimate of stiffness of tissue, such as actual displacement, or a related displacement characteristic. Displacement data may be a characteristic of actual displacement, such as strain rate, modulus or relaxation. Actual displacement indicates tissue relative stiffness and deformation. Strain rate indicates the first time derivative of the strain. Local strain rate may indicate cardiac muscle contractility from which is inferred the muscle health and condition. Modulus (e.g., Young's modulus) may be generated when the strain or strain rate is normalized by and combined with stress measurements. One method is to measure the pressure at the body surface with sensors attached to the transducer. The stress field pattern is then extrapolated internally to the points (i.e., pixels or voxels) of measured strain. Young's modulus is defined as stress divide by strain. Local modulus values may be calculated and those numerical values are converted to gray scale or color values for display.

The displacement data is determined from the two or more frames of ultrasound data representing the region under different levels of pressure or strain. The displacement of tissue in the region responsive to the displacement force is determined. One frame of ultrasound data represents the region prior to, after, or during application of the displacement force. Another frame of ultrasound data represents the region subject to a different amount of displacement. The displacement is determined as a function of the scans corresponding to different displacement pressures.

Any displacement function may be used. For example, B-mode data of the different frames is correlated along one, two, or three dimensions. An average, mean or other statistic of the directional correlation between the two frames of ultrasound data is determined. The displacement data is generated with one (e.g., M-mode), two (e.g., B-mode), three (e.g., static volumetric), or four (e.g., dynamic volumetric) dimensional acquisition and imaging. In one embodiment, any one or more of the methods or systems disclosed in U.S. Pat. Nos. 5,107,837; 5,293,870; 5,178,147; 6,508,768 or 6,558,324, the disclosures of which are incorporated herein by reference, are used to generate frames of displacement data.

For artifact reduction, two or more frames of displacement data are acquired. Acts 31, 32, 34, and 36 are repeated at least twice. The transducer is maintained in a same position for each repetition to acquire displacement data representing the same region.

The different frames of displacement data correspond to different angles, frequencies, and/or focus locations. For example, the scanning of act 34 is performed at two different transmit, receive, and/or transmit and receive frequencies.

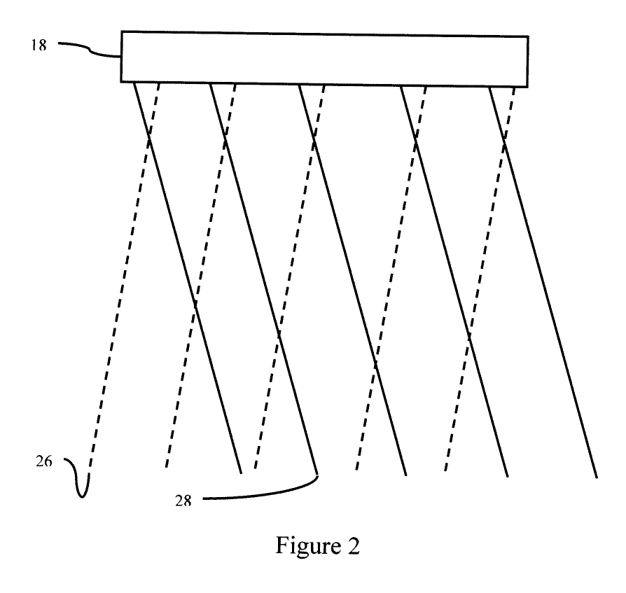

As another example, the displacement data of the different frames is responsive to different angles. The different angles apply to the direction of the displacement pressure and/or scanning. For example, FIG. 2 shows a transducer 18 with scan lines 26 and 28 at different angles to the transducer 18. The scan lines 26, 28 are used for applying acoustic force radiation and/or transmit and receive scanning. For different scanning angles, some of the region covered by one frame of displacement data is not covered by another frame of data due to the different angles. Sector or Vector® scanning may be used. The scan line angles for a given frame of data vary. The origin of the sector or Vector® scan is positioned differently for the different frames of displacement data. One or more angles with a same angle, but different origins, may be provided in the different frames of displacement data. The group of scan lines of each frame of displacement data use one or more angles. The groups of two different frames have one or more angles different than any of the one or more angles of the first group due to the origin difference. Each group consists of all the scan lines for the entire region associated with the corresponding frame of displacement data. The difference in angles results in different angles of scanning and/or application of displacement force for any given spatial location within the region.

FIG. 2 shows scanning at different angles. The displacement pressure is applied from a same angle or different angles for the different angle scans. In other embodiments, the displacement pressure is applied from different angles with scanning from the same or different angles. The displacement force originates at different locations corresponding to the different force angles relative to the region. For example, acoustic radiation force frames of displacement data are acquired with acoustic radiation force steered at the different force angles. The displacement may be from a same location and/or angle for scanning an entire frame.

In optional act 38, the frames of displacement data are normalized. One or more frames may be normalized relative to another frame. Alternatively, the different frames are each normalized. Any now known or later developed normalization of the tissue displacement frames of data may be used. For example, the amplitudes of the displacement data are normalized. An average or median of the displacement data of each frame is determined. An offset from a desired average or from the average of another frame is determined. The offset is added to the displacement data to equalize the average amplitude.

In another example, the dynamic range of the displacement data is updated. Each frame of displacement data may be a result of different compressions, changes in compression or other elasticity parameters. For the same tissue profile, two displacement profiles generated under two different compression force changes result in different dynamic ranges. Since displacement is a relative value, its number may not give easily used diagnosis information without knowing the stress.

To overcome the implicit drawback of the displacement, the dynamic range of the displacement data is updated. In most displacement applications, the region of interest (field of view) includes normal soft tissue, such as breast fat tissue, that can be used as the reference. The normal softest tissue has the highest displacement in the region of interest as compared with other normal and pathological tissue. According to Hook's law, the displacement is linearly proportional to the stress. This linear relationship is valid when the compression is small. The compression is small in practical applications for ultrasound. The ratio of the displacement in different tissues as a metric holds relatively constant although the displacement values may vary under different compression force.

To update the dynamic range, each frame of displacement data is normalized using the highest displacement value from the frame of displacement data or another frame of displacement data. For example, the maximum value of displacement is Eₘₐₓ. For each pixel (x,y), a displacement e(x, y) is determined. p(x, y) is the percentage calculated as e(x, y) divided by Eₘₐₓ. The color-coding or data used for imaging is based on the percentage value p(x, y), and the range of the color-coding is [α,1]. The percentage is mapped between α and 1. A value of 1 is the normal and most transparent in color, and α value of α is the most hard and red in color. The value α may be determined empirically from a set of pathological data.

After normalization, each frame of data has a similar dynamic range. In act 40, the frames of displacement data are combined. For example, normalized frames of displacement data associated with different angles are combined. Normalization may occur after combination.

For each of a plurality of spatial locations of the region of interest, the displacement data of different frames are combined. If a given frame does not include data representing the spatial location due to steering, the frame does not contribute to the combination for that spatial location. Scan converted data may be combined. Alternatively, data in a scan format is selected to represent a given spatial location by interpolation, extrapolation, or nearest neighbor selection. Displacement data for each spatial location in the region is combined. Displacement data of the tissue displacement frames representing the same locations and responsive to the different displacement force angles are compounded. Any combination function may be used, such as averaging, weighted averaging, maximum selection, minimum selection, median selection, or other now known or later developed combinations.

In act 42, an image is generated from the combined frames of data. The combined displacement values are output for display. For example, the displacement values are mapped with a grayscale or color map. Other information may be added. For example, a color map is selected for displacement data and a gray scale map is selected for B-mode data. A common map outputting display values for a linear or nonlinear combination of displacement and other data may be provided.

The image represents the displacement in the region of interest. The image is a function of the tissue displacement frames of data. Combined displacements for each spatial location are provided for the image. Images may be updated as more frames of displacement data with the same or different angles are obtained. Each new frame is added to the combination or the combination is formed from frames selected by any window function. The image or the combination without image mapping may be stored for later image generation.

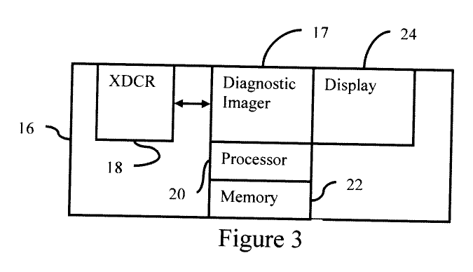

FIG. 3 shows one embodiment of a system 16 for ultrasound-based displacement imaging with reduced artifacts. The system 16 implements the method of FIG. 1 or other methods. The diagnostic imaging system 16 includes a diagnostic imager 17, a transducer 18, a processor 20, a memory 22, and a display 24. Additional, different or fewer components may be provided. For example, the processor 20 and/or memory 22 are separate from the imaging system 16. As another example, a user input is provided for manual or assisted selection of view parameters or other control. In another embodiment, the system 16 is a personal computer, workstation, PACS station, or other arrangement at a same location or distributed over a network for real-time or post acquisition imaging, and does not include the transducer 18.

The transducer 18 is an array of elements. One, two or multi-dimensional arrays may be used. Piezoelectric or cMUTs may be used. The transducer 18 is sized and shaped for transmission and reception of diagnostic ultrasound, such as acoustic energy with relatively low intensity. The transducer 18 converts between acoustic and electrical energies for scanning and/or applying acoustic displacement force. Switches or other components may be provided for selecting different apertures for transmission or reception at different angles.

In one embodiment, the transducer 18 is in a handheld housing. The handheld housing may be used to apply the displacement pressure. Alternatively, one or more components built into the handheld housing or separate from the housing are used to apply the displacement pressure. For example, a moveable plate or transducer is provided at each end of the transducer in the housing. The user or a motor causes the plates to exert a pressure on the skin of the patient. The spatial distribution provides different angles of applied pressure relative to the region.

The diagnostic imager 17 includes a beamformer, a detector (e.g., B-mode and/or Doppler), a scan converter, and a display. Additional, different or fewer components may be provided, such as including filters. The diagnostic imager 17 generates transmit waveforms for scanning with the transducer 18. The transmit waveforms may be high amplitude for acoustic force radiation or relatively lower amplitude for scanning. The transducer 18 converts echoes into electrical signals for beamformation by the imager 17. The beamformed data is detected and used for imaging. In one embodiment, the imager 17 includes a B-mode detector operable to generate B-mode or intensity data in response to the echoes. In another embodiment, the imager 17 includes a Doppler detector operable to estimate velocities or other tissue movement in response to the echoes. The imager 17 includes any now know or later developed components for implementing any displacement, elasticity, or ARFI imaging. In other embodiments, a therapy system is provided and used for generation of acoustic radiation force.

The processor 20 is a control processor, general processor, digital signal processor, application specific integrated circuit, field programmable gate array, graphics processor, Doppler processor, digital circuit, analog circuit, combinations thereof, or any other now known or later developed device for determining displacement or correlating. The processor 20 is part of the imager 17, but may be part of a separate system. The processor 20 controls operation of the imager 17.

Alternatively or additionally, the processor 20 determines strain or displacement as a function of echoes. The imager 17 transmits a sequence of pulses, such as diagnostic pulses. Data detected from responsive echoes are used to determine displacement. Displacement may be determined as a function of the displacement of tissue. In one embodiment, the processor 20 correlates B-mode data from different transmit events. By searching for a best or sufficient fit in one, two, or three dimensions, an amount of displacement between the different transmit events is determined. In another embodiment, Doppler estimates are generated from echoes generated from different transmit events. For example, velocity is estimated. The velocity and time may be used to determine a displacement. Alternatively, displacement is directly estimated based on the velocity. The processor 20 determines the displacement for a plurality of spatial locations at least twice, with the displacement of each frame being associated with a different scan or displacement force direction.

The memory 22 is a computer readable storage medium, such as a cache, buffer, register, RAM, removable media, hard drive, optical storage device, or other computer readable storage media. Computer readable storage media include various types of volatile and nonvolatile storage media. The memory 22 is part of the imager 17, the imaging system 16, or separate from both. The memory 22 is accessible by the processor 20.

In one embodiment, the memory 22 stores data for use by the processor 20, such as storing detected and/or image data for determining displacement. Additionally or alternatively, the memory 22 stores data representing instructions executable by the programmed processor 20 for ultrasound-based displacement imaging with reduced artifacts. The instructions for implementing the processes, methods and/or techniques discussed herein are provided on computer-readable storage media or memories. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for ultrasound-based displacement imaging with reduced artifacts, the method comprising:
acquiring a first frame of displacement image for a first region corresponding to a first position of a transducer, the displacement data of the first frame responsive to a first angle;
acquiring a second frame of displacement data with ultrasound for the first region corresponding to the first position of the transducer, the displacement data of the second frame responsive to a second angle different than the first angle;
combining, for each of a plurality of spatial locations of the first region, the displacement data of the first frame with the displacement data of the second frame; and
generating an image of the first region as a function of the combined displacement data.

2. The method of claim 1 wherein acquiring the first frame of displacement data comprises scanning while applying different amounts of pressure with the transducer against a patient while the transducer is maintained in the first position and wherein acquiring the second frame of displacement data comprises scanning while applying different amounts of pressure with the transducer against the patient while the transducer is maintained in the first position.

3. The method of claim 1 wherein acquiring the first and second frames of displacement data comprises applying acoustic pressure in the first region and scanning with and without the acoustic pressure.

4. The method of claim 1 wherein acquiring the first and second frames of displacement data each comprise determining a second correlation between the two or more frames of ultrasound data associated with different pressures in the first region.

5. The method of claim 1 further comprising:
normalizing the displacement data of the first frame; and
normalizing the displacement data of the second frame;
wherein combining comprises combining as a function of the normalized first and second displacement data.

6. The method of claim 1 wherein acquiring the first and second frames of displacement data comprises scanning with different steering angles corresponding, at least in part, to the first and second angles, respectively.

7. The method of claim 1 wherein acquiring the first and second frames of displacement data comprises applying displacement pressure from different angles relative to the first region, the different angles corresponding to the first and second angles, respectively.

8. The method of claim 7 wherein acquiring the first and second frames of displacement data comprises scanning with different steering angles corresponding, at least in part, to the first and second angles, respectively.

9. The method of claim 1 wherein acquiring the first and second frames of displacement data comprises scanning at first and second different scanning frequencies, respectively.

10. In a computer readable storage medium having stored therein data representing instructions executable by a programmed processor for ultrasound-based displacement imaging with reduced artifacts, the storage medium comprising instructions for:
forming tissue displacement frames of data in response to different displacement force angles, the tissue displacement frames of data representing a same region; and
generating an image of the region as a function of the tissue displacement frames of data.

11. The computer readable storage medium of claim 10 wherein forming tissue displacement frames of data comprises forming elastography frames of data with an external force source at different locations corresponding to the different force angles relative to the region.

12. The computer readable storage medium of claim 10 wherein forming tissue displacement frames of data comprises forming acoustic radiation force frames of data with acoustic radiation force steered at the force angles.

13. The computer readable storage medium of claim 10 wherein forming tissue displacement frames of data comprises correlating ultrasound data responsive to tissue subject to different amounts of displacement force.

14. The computer readable storage medium of claim 10 further comprising instructions for normalizing the tissue displacement frames of data.

15. The computer readable storage medium of claim 10 wherein generating an image of the region as a function of the tissue displacement frames of data comprises compounding displacement data of the tissue displacement frames representing the same locations and responsive to the different displacement force angles.

16. A method for ultrasound-based displacement imaging with reduced artifacts, the method comprising:
positioning a transducer adjacent a region to be imaged;
transmitting first acoustic force from the transducer at a first group of one or more angles relative to the transducer;
determining first displacement of tissue in the region responsive to the first acoustic force;
transmitting second acoustic force from the transducer at a second group of one or more angles relative to the transducer, the one or more angles of the second group different than any of the one or more angles of the first group;
determining second displacement of the tissue in the region responsive to the second acoustic force;
combining, for each spatial location in the region, the first and second displacements, the first and second groups corresponding to scan lines for scanning the entire region; and
generating an image of the region as a function of the combined first and second displacements for each spatial location.

17. The method of claim 16 wherein each determining comprises:
scanning the region prior to transmitting;
scanning the region after transmitting; and
determining the displacement as a function of the scans.

18. The method of claim 16 wherein combining comprises averaging.

19. The method of claim 16 wherein the first and second groups consist of scan lines for applying the first and second acoustic forces, respectively, for the entire region.

20. The method of claim 16 further comprising:
maintaining the transducer at a same position for the transmitting and determining acts.
